(19) United States
(12) Patent Application Publication
(10) Pub. No.: US 2011/0213218 A1
(43) Pub. Date: Sep. 1, 2011

(54) Patient healthcare monitoring/maintenance system

(76) Inventors: Bert A. Weiner, Thousand Oaks, CA (US); Mark S. Goodman, Needham, MA (US)

(21) Appl. No.: 12/928,794

(22) Filed: Dec. 17, 2010

Related U.S. Application Data
(60) Provisional application No. 61/284,471, filed on Dec. 17, 2009.

(51) Int. Cl. A61B 5/00

(52) U.S. Cl. 600/301

(57) ABSTRACT

An independently operable interactive health care monitoring and control system for the patient-responsive treatment and reporting from a home-based patient with respect to a health care provider treatment facility, the system comprising a patient or system activated “body-condition” test unit configured for personal recognition and interactive telecommunication with the health care provider treatment facility; and a patient-personalized, screen displayed, patient-respected computer generated avatar figure, to prompt and guide proper patient evaluation, reporting, behavior, behavior modification and treatment.

### BACKGROUND OF THE INVENTION

1. Field of the Invention

This invention relates to patient health-monitoring systems and more particularly to systems using patient-developed avatar-arrangements for encouraging patient medical care-requirement-compliance, including arrangements for patient-reminding, overseeing, advising and communicating between medical personnel and patient, about a patient's health condition, and is based upon Provisional Patent Application Ser. No. 61/284,471, filed 17 Dec. 2009, and incorporated herein by reference, in its entirety.

2. Prior Art Discussion

The field of patient monitoring is evolving as the need for health care is growing. There are currently over 110 million Americans having chronic illnesses who would benefit from an improved home based monitoring arrangement to facilitate improved healthcare.

The old standby of visiting nurses attending a patient's home, needs to be updated by the use of modern telecommunications. Telemedicine has been attempted over a period of years but has several shortcomings. Such telecommunication is common in hospital settings and emergency rooms and clinics, yet complete systems with home-based patient's still fall short of the desired level of service.

There is a need for a convenient accurate, home-based, patient-accessible and patient-interactive audiovisual communication system for patient monitoring and for patient-needs fulfillment connected between the patient and the caregiver.

There is also a need for a “friendly” and yet “demanding” patient “reminding” or patient “encouraging” system for motivating a patient to take the proper action when necessary, and to notify appropriate medical personnel when such action is not been undertaken.

### BRIEF SUMMARY OF THE INVENTION

The present invention comprises a system for interacting with a patient in a home based patient-caregiver health monitoring and support system. The health monitoring and support system is arranged to be patient activated, or preferably automatically and independently, system-activated, self-administrated diagnostic procedures of, for example: blood pressure, pulse oxygen, blood glucose and body temperature. Once these self administered procedures have been accomplished by the patient, direct audiovisual communication between the healthcare provider and the patient is permitted/encouraged by the present system.

This regular support and mentoring/monitoring by the system, particularly of chronically ill or absent-minded patients, utilizes personality and character-individualized, specially created or specifically selected, highly patient-responsive, “atavaristic” reminders and persuaders to affect the patient to take his/her medications and use proper behavioral treatment. The system additionally, provides historic information on the patient's accumulated tests to date, to follow trends and events of that patient and his or her proper following of medical procedures.

The system is contained in a unitary primary structure or housing, for example, at bedside use within a patient's home, with satellite housings in wired or wireless communication with the primary housing, to continue a home-wide prompting, encouraging and reminding the patient, by one or more specifically favored or created avatars to enforce proper behavior and medication utilization. The system housing includes among other test and reporting devices and procedures, a blood pressure monitor test unit with its affiliated blood pressure cuff and blood pressure analysis circuit with input to the patient's telecommunication control unit.

The system also includes a pulse oxygen clip test unit. The pulse oxygen clip (oximeter) is in communication with the systems analysis circuit with input to the patient's telecommunication control unit, in timed, preferably sequentially programmed use with the blood pressure cuff.

The system also preferably includes a body thermometer test unit such as for example an ear thermometer. The body thermometer is in communication with the system's analysis circuit with input to the patient's telecommunication control unit.

The system may also include a glucometer test unit for receiving patient acquired blood test strips. The glucometer is in communication with the systems analysis circuit with input to the patient's telecommunication control unit.

The telecommunication control unit includes a patient and healthcare provided controllable video monitor with a touch screen on which instructions and preferred variable avatars appear as needed. Speakers adjacent the monitor may provide preprogrammed sound effects and/or verbal instructions to the patient. The monitor thus provides the desired, specially created and/or selected (variable) avatar for patient encouraging, motivating and instructional purposes. The video monitor and associated touch screen is arranged to instruct and receive responses from the patient relative to the various test units and their step wise procedures. The prompts generated by the avatars and received by the patient and the patient's responses to the selected avatar's prompts for appropriate test unit initiatives are communicated to the patient's healthcare provider preferably via the Internet or may be pre-programmed so as to present one or more of a range of variable “avatar personalities” desired or needed to get the point across and motivate the patient into proper behavior.

If the patient's responses are inadequate or the test unit results fall outside of the desired healthcare providers parameters, the inventive system and/or healthcare provider may automatically make changes to the avatar's character and persuasiveness needed for fulfilling the patient's required medical procedures or medicaments. Audio visual communication in addition to verbal or oral discussion may also be instituted between the patient and the healthcare provider upon certain test results communicated to the healthcare provider or upon failure of the patient to properly respond to or actually perform certain test units from the particular avatar or system prompts.

The system's use preferably begins with usage of the pulse oxygen clip being initiated when, in one aspect of the invention, the patient first puts his arm through the blood pressure cuff opening in the front of the housing. Once the arm is properly through the blood pressure cuff opening, the patient's fingers will be able to reach the pulse oxygen finger clip (oximeter). The patient is then instructed by the programmed avatar on the monitor to slide his index finger into the clip. A “ready light” may be activated by an actuation trigger mechanism in communication with the monitor when the finger is fully entered into the clip. The patient is then instructed by the personalized, patient-respected avatar on the monitor, to touch the screen when the patient is ready for the pressure and pulse oxygen measurement to begin. The pulse oxygen clip functions within the index finger and the tests results appear on the touch screen. A test complete display light preferably appears on the monitor and the results are forwarded to the telecommunications portion of the system for further reporting to the healthcare provider as necessary.

Once a good pulse waveform has been received by the system, the blood pressure monitor test unit procedure begins, which procedure comprises the patient keeping his arm through the opening within the test unit, the system's proper computer automatically activating the appropriate inflation switch, preferably after an avatar greeting and any necessary prompting, wherein the inflatable pressure cuff blows up and closes around the arm or wrist area holding that cuff tight to the patient. A sensor arrangement in the cuff reads the patient's blood pressure and corroborates the heart rate picked up by the pulse oximeter. The blood pressure monitor test unit preferably includes an LED light display showing, when flashing, that a test is in progress. The electronic data received by the blood pressure monitor test unit is sent through the telecommunications unit to the healthcare provider's website for data storage, and/or to an alert at the medical providers' facility for a doctor's attention, or for a stronger, more detailed or aggressive message or instruction by a possibly character-changed avatar. A repeat pulse oximeter cross-check may be performed after the blood pressure analysis has been finished, to establish pattern accuracy, the reminder for such cross-check being prompted by the avatar, as necessary.

A further body condition test, such as for example, a body thermometer, such as an ear thermometer, is accomplished by insertion of, for example, a cone shaped ear piece into the ear canal from its support on the housing, prompted as needed by an avatar on one or more of the system monitors within the home. The touch screen on the monitor may be automatically actuated by the displacement of the ear thermometer from its support in the housing enclosure triggering a response through a proper circuit within the monitor. The patient presses the appropriate indicated location on the monitor's touch screen and LED lights indicate that the test is underway and or completed. The electronic data received by the body thermometer test unit is sent through the telecommunications unit to the healthcare provider's website for data storage or to alert someone at the medical provider's facility for a doctor's attention. The patient may be advised by an appropriate avatar on his monitor whether to take/follow certain medical procedures or not, depending upon the results of the particular test.

A glucometer may be initiated when the personalized avatar on the screen monitor prompts the patient to slide a glucose test strip into a test strip opening of the glucose test unit arranged within the housing of the system. The test strip opening preferably includes a trigger sensor which is activated when a test strip is inserted therein. The monitor then receives the triggered sensor's signal and effects a notice on the touch screen monitor for the patient to put his finger in place and deposit a droplet of blood onto the test strip. The glucometer meter then is instructed by the control unit to read the test strip and the glucose level results appear on the monitor's touch screen wherein LED lights signal that the test is complete. The test results are then automatically sent to the telecommunications unit of the system and to the healthcare provider for review if parameters so indicate.

The test results will also be presented to the patient by the avatar or displayed on the touch screen as appropriate graphical data so as to encourage the patient to maximize his self care and for him to reach the desired health care goals. The “goal” aspect will relate to improvement or deterioration information which will be utilized to encourage the patient to maximize his self-care, with the possibility of system wide interaction and/or competition with others of his demographic or his diagnosis. A further aspect of this procedure may be that prizes may be awarded by the healthcare provider, such as spa treatments or premium reductions for insurance or the like, to encourage healthy behavior. Such a data generated by such information may be able to be used for analysis of effectiveness of the healthcare treatment.

The incrementally variable or personality-advanceable or personality-selectable “avatar”, a critical element of the invention, may appear in the form of the patient's physician, another person of authority, a representation of a member of the patient's family, a sports figure, a friend, a political figure, a historical figure, or a cartoon character, or the like, so as to induce the patient to be particularly responsible taking the tests and following though with the touch screen prompts. The avatar may be programmed or selected for various multiple levels of encouragement or persuasion, becoming more friendly or stern as needed depending upon the timing or non-completion of the patient's desired/required response.

The invention thus comprises a health care monitoring and control system for the patient-responsive treatment and reporting from a home-based patient with respect to a health care provider treatment facility, comprising: a patient activated or system activated “body-condition” test unit configured for personal recognition and interactive telecommunication with the health care provider treatment facility; and a patient-personalized, screen displayed, patient-respected computer generated avatar figure, to prompt and guide proper patient treatment. The “body-condition” test unit may comprise a test unit for measuring patient self-administered blood oxygen levels. The “body-condition” test unit may comprise a test unit for measuring patient self-administered blood pressure. The “body-condition” test unit may comprise a test unit for measuring patient self-administered body temperature. The “body-condition” test unit comprises a test unit for measuring patient self-administered glucose levels. The avatar figure is preferably generated by a computer source within the monitoring system or an interne source. The computer source preferably maintains a variable avatar collection to permit an avatar to show various or multiple stages of urgency to a patient utilizing the system, as required when patient compliance is not properly completed with certain preselected time intervals. The avatar is preferably selected from a group consisting of: one or more of the patient's family members, the patient's medical support team, a religious figure, a friend, a historical figure, a sport's figure, and a cartoon character.

The invention also may comprise a method of providing health care monitoring and health care support by a home based monitoring and healthcare support system comprising: arranging a patient-specific, patient-personalized, avatar-pre-programmed healthcare monitoring and control system for in-home use by a specific health impaired patient; initiating the system monitoring and support of a patient using the system by introduction of a patient's limb through a patient-receiving annular blood pressure monitoring cuff; guiding the patient through the monitoring and support procedure by a patient-identifiable avatar pre-selected by the patient/healthcare provider, and represented on a monitor of the system; wherein the patient identifiable avatar is pre-selected by the patient from the group consisting of: one or more of the patient's family members, the patient's medical support team, a religious figure, a historic figure, a sport's figure, and a cartoon character, and wherein the healthcare monitoring system is in electronic communication and monitored by a professional healthcare agency; testing the patient's blood by a finger clip oximeter for an oximeter reading prior to reading the patient's blood pressure by the blood pressure monitoring cuff; testing the patient's blood pressure subsequent to the oximeter reading as prompted and instructed by the particular avatar character; reporting the results of the system's testing to a healthcare provider via an internet communication therewith; elevating the urgency expressed by the avatar in communication with the patient undergoing the test procedures if the system or the healthcare provider determines an improper or undesired result or condition has occurred; changing the avatar from one initially utilized to a different more forceful avatar should the system indicate a need to do so by its computer; and changing the avatar from one initially utilized by the system and shown on a monitor to the patient, to a different more forceful avatar shown subsequently to the patient on a monitor, should the system indicate a need to do so by the healthcare provider.

The invention may also comprise a method of providing health care monitoring and health care support by a home based monitoring and healthcare support system in the house of a patient, comprising: arranging a patient-specific, patient-personalized, avatar-pre-programmed healthcare monitoring and control system for in-home use by a specific health impaired patient; initiating the system monitoring and support of a patient using the system, by introduction of a patient's limb through a patient-receiving annular blood pressure monitoring cuff; and guiding the patient through the monitoring and support procedure by a patient-identifiable, avatar pre-selected by the patient/healthcare provider, and represented on a monitor of the system, wherein the patient identifiable avatar is pre-selected by the patient from the group consisting of: one or more of the patient's family members, the patient's medical support team, a religious figure, a historic figure, a sport's figure, and a cartoon character, and wherein the healthcare monitoring system is in electronic communication and monitored by a professional healthcare agency, and the steps of: testing the patient's blood by a finger clip oximeter for an oximeter reading prior to reading the patient's blood pressure by the blood pressure monitoring cuff; testing the patient's blood pressure subsequent to the oximeter reading as prompted and instructed by the particular avatar character; reporting the results of the system's testing to a healthcare provider via an internet communication therewith; elevating the urgency expressed by the avatar in communication with the patient undergoing the test procedures if the system or the healthcare provider determines an improper or undesired result or condition has occurred; changing the avatar from one initially utilized, to a different more forceful avatar should the system indicate a need to do so by the patient's response sensed by the system computer, or changing the avatar from one initially utilized by the system and shown on a monitor to the patient, to a different more forceful avatar shown subsequently to the patient on a monitor, should the system indicate a need to do so by the healthcare provider, and wherein the system includes satellite monitors selected from the group consisting of: wireless monitors in further rooms within the house of the patient, wireless monitors worn by the patient for receipt of avatar instruction.

### DESCRIPTION OF FIGURES FOR PRESENT PATIENT ACTUATED HEALTH CARE SYSTEM

The objects and advantages of the present system will become more apparent when viewed in conjunction with the following drawings, in which:

FIG. 1 is a diagrammatic representation of the patient actuated health care system of the present invention;

FIG. 2 is a flowchart representing the general outline of the patient actuated health care system shown in FIG. 1;

FIGS. 3A, 3B, 3C and 3D are flowcharts representing more particularly the procedures of the patient actuated health care system as it relates to a blood pressure monitoring arrangement, a body so monitor each of which being in communication with the telecommunications unit including the touch screen monitor;

FIG. 4 is a flowchart representing more particularly the procedures of the patient actuated health care system as it relates to blood pressure monitoring, ear temperature monitoring, blood oxygen clip testing, and glucose monitoring by a glucometer; and

FIG. 5 is a general flowchart representing the procedures utilized by a patient with the present health care monitoring system when that patient is released from a hospital and needs subsequent healthcare monitoring and guidance.

### DETAILED DESCRIPTION OF THE INVENTION

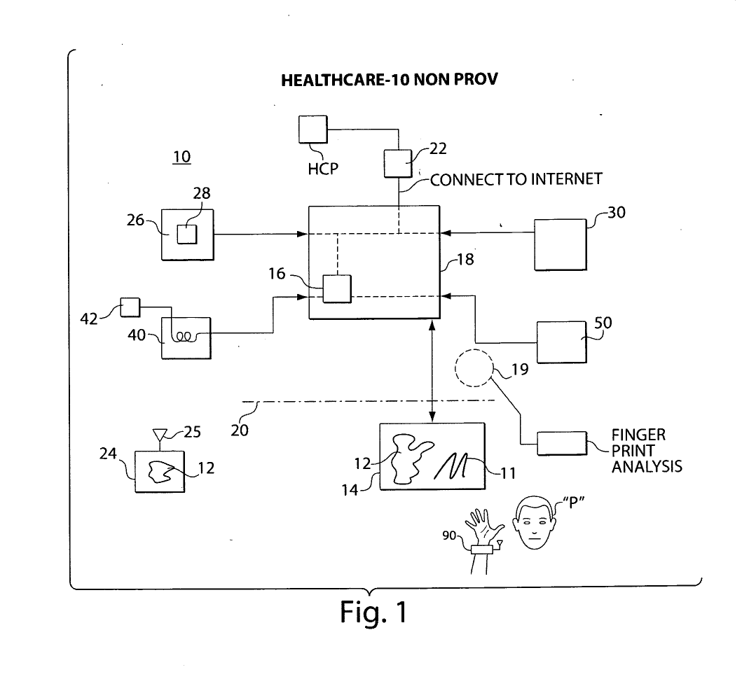

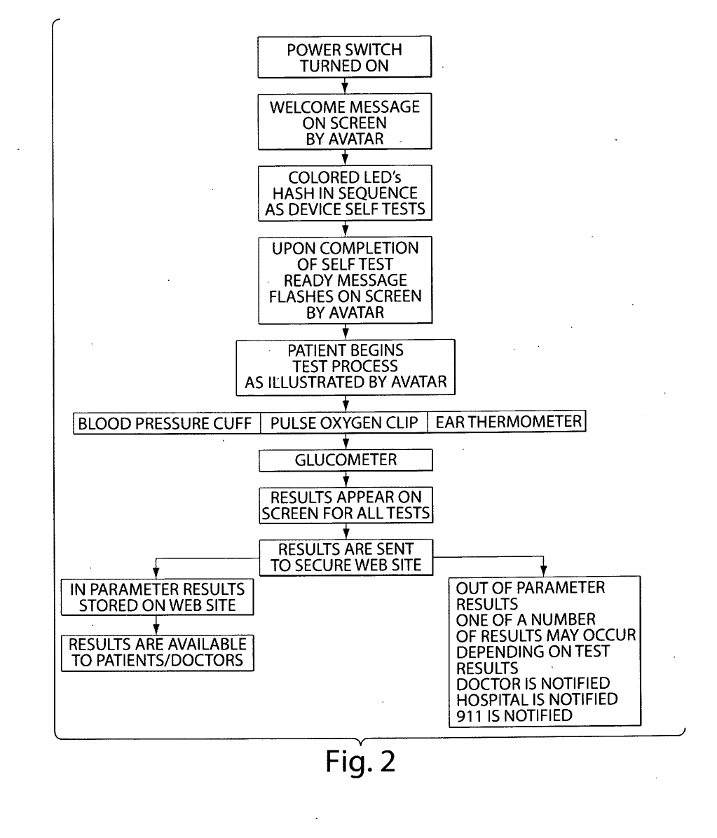

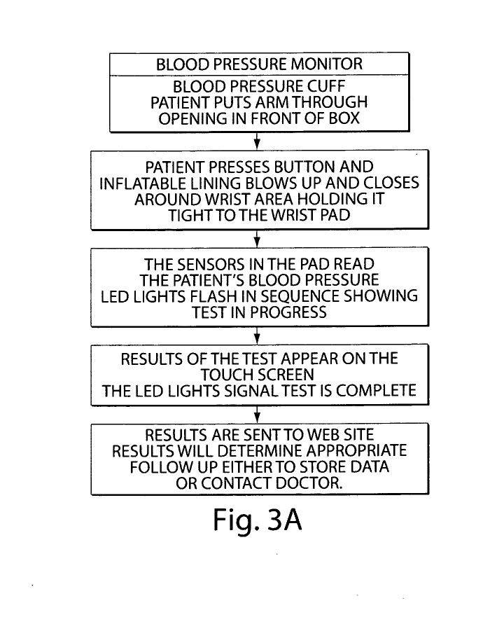

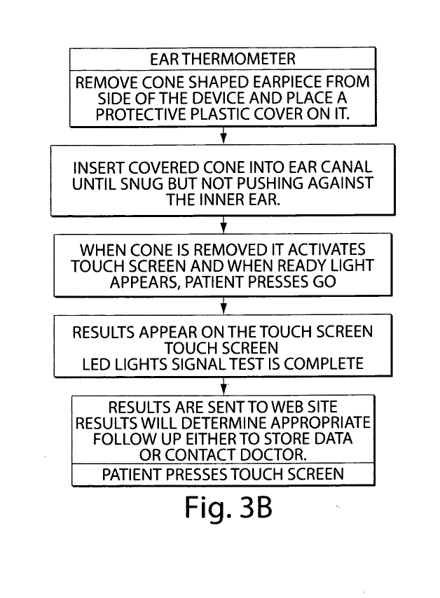

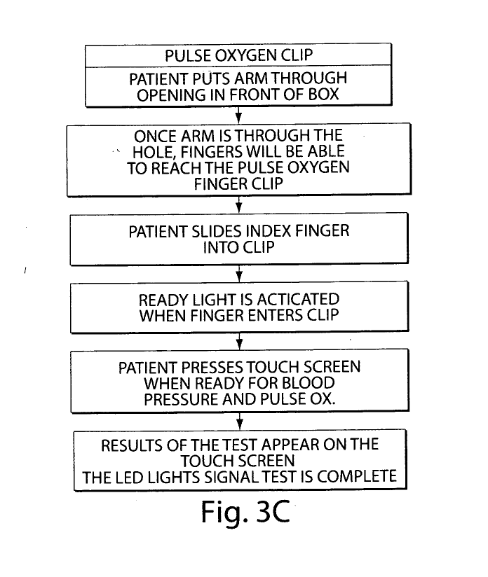

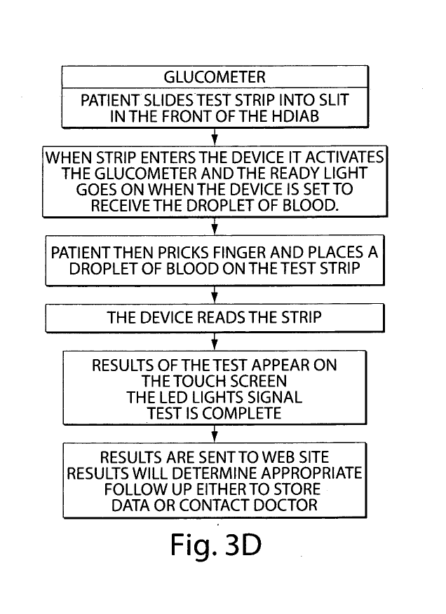

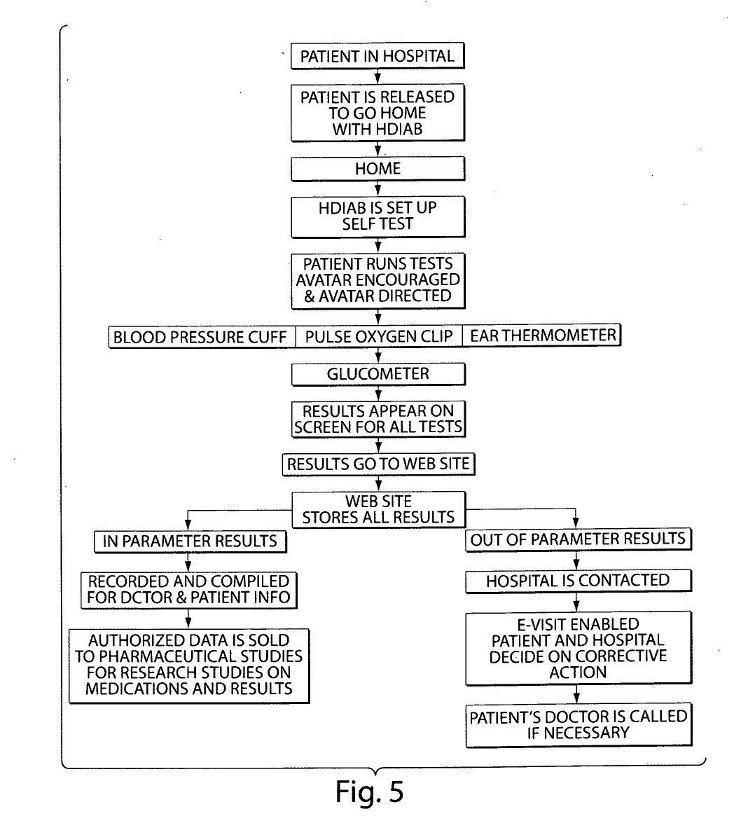

Referring now to the drawings in detail and particularly to FIG. 1, there is shown the present invention in which a system for interacting with a patient “P” in a home based patient-caregiver health monitoring and support system 10. The health monitoring and support system 10 is arranged to be patient activated, or preferably automatically and independently system activated for the patient's self-administrated diagnostic procedures of, for example: blood pressure, pulse oxygen, blood glucose and body temperature, as represented in FIG. 2, and represented more specifically in FIGS. 3A, 3B, 3C, 3D, 4 and 5. Once these self administered procedures have been accomplished by the patient, with the assistance of personalized, preselected, action-encouraging avatars 12, which are displayed on a patient's touch screen 11 on the monitor 14, direct audiovisual communication between the health care provider HCP and the patient “P” is permitted/encouraged by the present system 10.

This regular support and mentoring/monitoring by the assembled system 10, represented schematically in FIG. 1, particularly for chronically ill or absent-minded type patients, utilizes any of a wide variety of those avatars 12, which are personality and character-individualized, specially created or specifically selected, highly patient-responsive, “atavaristic” reminders and persuaders to affect the patient to take his/her medications and use proper behavioral treatment. Those selectable, various situation-adaptable avatars 12, are preferably generated from previous patient inquiries or patient histories, or created by proper software within the system 10 or by the health care provider HCP especially for the psychological nature of the particular patient, the avatars 12 preferably being stored within and retrievable from a proper computer memory 16 in the control unit 18, in a primary housing 20, or otherwise may be modified as needed, and/or retrieved from a proper on-line internet source 22. Each selectable pre-loaded or on-line available avatar 12 preferably has its own distinctive sound or audio presentation which is known to correspond to the particular avatar 12 (i.e.: friend, doctor, family member, sports figure etc.) selected by the patient P or by the computer 16 and/or control unit 18. The system 10 additionally, provides historic information on the patient's accumulated tests to date, to follow trends and events of that patient and his or her proper following of medical procedures.

The system 10 is contained in a unitary primary structure or housing 20, for example, at bedside use within a patient's home, with satellite housings 24 in wired or a wireless antenna 25 communication with the primary housing 20, to continue a home-wide prompting, encouraging and reminding the patient, by one or more specifically favored or created avatars 12 to enforce proper behavior and medication utilization. The system housing 20 includes among other test and reporting devices and procedures, a blood pressure monitor test unit 26, shown in FIG. 1, the operational steps being represented in FIG. 3A, with its affiliated blood pressure cuff and blood pressure analysis circuit 28 with proper input to the patient's telecommunication control unit 18. A further aspect of the system 10 may include a wireless unit 90, such as a radio frequency wrist watch-like prompter device carried by the patient on his person, to remind the patient by a miniaturized avatar with its respective audio admonition, to take his medications or to submit to testing via the system 10.

The system 10 also preferably includes a pulse oxygen clip test unit 30. The pulse oxygen clip (oximeter) 30 is in communication with the systems analysis circuit computer 16 through input to the patient's telecommunication control unit 18, in timed, preferably sequentially programmed use with the blood pressure cuff.

The system 10 also preferably includes a body thermometer test unit 40 such as for example an ear thermometer 42. The body thermometer 40 is in communication with the system's proper analysis circuit computer 16 though input to the patient's control unit 18 telecommunications.

The system 10 may also include a glucometer test unit 50 for receiving patient acquired blood test strips, not shown for clarity of viewing. The glucometer 50 is in communication with the systems computer analysis circuit 16 through input to the patient's control unit 18 telecommunications.

The control unit 18 telecommunication arrangement includes the patient and healthcare provided controllable video monitor 14 (and satellite unit(s) 24) with a touch screen 11 on which the medical instructions, and the “preferred” avatars 12 appear as needed. Speakers (not shown) adjacent or with the monitor 14 may provide preprogrammed sound effects and/or verbal instructions to the patient. The monitors 14 and 24 thus provide the desired, specially created and/or selected avatar 12 for patient encouragement, motivation and instructional purposes. The video monitor 14 and associated touch screen 11 is arranged to instruct and receive responses from the patient “P” relative to the various test units 26, 30, 40 and 50, represented in FIG. 1, and their step wise procedures represented in FIGS. 3A, 3B, 3C and 3D.

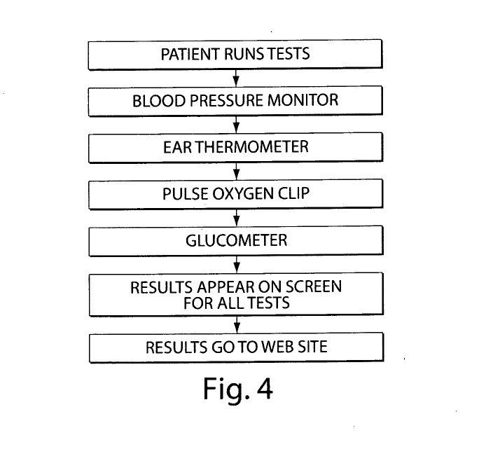

The prompts generated by the avatar(s) 12 and received by the patient “P”, and the patient's responses to the selected avatar's prompts for appropriate test unit initiatives are communicated to the patient's health care provider HCP preferably via the Internet as represented stepwise in FIGS. 1 and 4. The computer 16 within the control unit 18 may be pre-programmed so as to present one or more of a range of “avatar personalities” desired or needed to get the point across and motivate the patient “P” into his/her proper medical behavior.

If the patient's responses are inadequate or the test unit results fall outside of the desired health care provider's parameters, the inventive system 10 and/or health care provider HCP may automatically make computer generated changes to the character of the avatar 12 (i.e. pushyness, domineering forcefulness or persuasiveness) needed for getting the patient to fulfill the patient's required medical procedures or take medicaments. Audio visual communication in addition to verbal or oral discussion may also be instituted between the patient and the healthcare provider upon certain test results communicated to the health care provider or upon failure of the patient to properly and timely respond to or actually perform certain test units from the particular avatar 12 or system prompts.

The system's use preferably begins with usage of the pulse oxygen clip 30 being initiated when the patient first puts his arm or limb through the arm or limb cuff opening 19 in the front of the housing 20. Once the arm is through the cuff opening 19, the patient's fingers will be able to reach the pulse oxygen finger clip 30 (oximeter). The patient is then automatically instructed by the programmed avatar 12 on the monitor 14 to slide his index finger into the clip oximeter 30. A “ready light” (not shown for clarity of the figures) may be activated by a proper actuation trigger mechanism in communication with the monitor 14 when the finger is fully entered into the clip oximeter 30. The patient “P” is then instructed by the personalized, patient-respected avatar 12 on the monitor 14 or 24, (or 90), to touch the screen 11 when the patient is ready for the pressure and pulse/oxygen measurement to begin. The pulse oxygen clip 30 functions with the index finger and the tests results appear on the touch screen. A test complete display light appears on the monitor 14 and the results are forwarded to the telecommunications portion of the system 10 for further reporting to the health care provider HCP, as necessary.

Once a good pulse waveform has been received by the system 10, the blood pressure monitor test unit's 26 procedure then begins, which procedure comprises the patient keeping his (or her) limb or arm through the opening 19 within the test unit housing 20, the system automatically initiating or the patient pressing the appropriate inflation switch (not shown for clarity of the figures) wherein the inflatable pressure cuff blows up and closes around the limb or wrist area holding that cuff tight to the limb or wrist. A sensor arrangement in the pad 28 reads the patient's blood pressure and corroborates the heart rate first picked up by the pulse oximeter 30. The blood pressure monitor test unit 26 preferably includes a proper circuit of an LED light display, showing, when flashing, that a test is in progress. The electronic data received by the blood pressure monitor test unit 26 is sent through the computer 16 in the telecommunications unit to the health care provider's website for data storage, and or to an alert at the medical providers' facility for a doctor's attention at a professional healthcare provider's facility (doctor's office/hospital), or for a stronger, more detailed or aggressive message or instruction by a possibly “character-changed” and newly emboldened avatar 12 from the proper data source in the computer or pre-determined on-line source. A repeat pulse oximeter cross-check may be performed after the blood pressure analysis has been finished, to establish pattern accuracy, the reminder for such cross-check being prompted by the avatar, as necessary.

A body thermometer, for example, an ear thermometer 42, may be accomplished by insertion of, for example, a proper cone shaped ear piece into the ear canal from its support on the housing 20, prompted as needed by an avatar 12 on one or more of the system monitors 14 or 24 within the home. The touch screen 11 on the monitor 14 may be automatically actuated by the displacement of the ear thermometer from its support in the housing enclosure, triggering a response through a proper circuit within the monitor 14. The patient presses the appropriate indicated location on the touch screen 11 of the monitor 14, and proper LED lights (not shown for clarity of viewing) may be arranged to indicate that the test is underway and or completed. The electronic data received by the body thermometer test unit 40 is properly sent through the telecommunications unit computer 16 to the website of the health care provider HCP for data storage or to alert someone at the medical provider's facility for a doctor's attention. The patient P may be advised by an appropriate (new or patient-familiar) avatar 12 on his monitor 14 or 24 whether to take/follow certain medical procedures or not, depending upon the results of the particular test.

A glucometer 50, represented in FIG. 1, may be initiated when the personalized avatar 12 on the screen 11 of the monitor 14 prompts the patient P to slide a glucose test strip into a test strip opening (not shown for clarity of the figures) of the glucose test unit arranged within the housing 20 of the system 10. The test strip opening preferably includes a trigger sensor which is activated when a test strip is inserted therein. The monitor 14 then receives the triggered sensor's proper signal and effects a notice on the touch screen 11 of the monitor 14 for the patient “P” to put his finger in place and deposit a droplet of blood onto the test strip. The glucometer unit 50 then is instructed by the computer 16 in the control unit 18 to read the test strip and the glucose level results appear on the touch screen 11 of the monitor 14 wherein a proper circuit of LED lights (not shown for clarity of viewing) signal that the test is complete. The test results are then automatically sent through the computer 16 of the telecommunications unit 18 of the system 10 and to the health care provider HCP for review if parameters so indicate.

The “avatar” 12 may be computer generated so as to appear in the form of the patient's physician, or another person of authority, or possibly a representation of a member of the patient's family, a member of the clergy, a sports figure or political figure, or a cartoon character, so as to induce the patient P to be particularly responsible taking the tests and following though with the touch screen prompts. The avatar 12 may also be programmed for multiple levels of encouragement or persuasion, becoming more friendly or stern as needed, depending upon the timing and/or non-completion of the patient's desired/required response.

We claim:

1. A health care monitoring and control system for the patient-responsive treatment and reporting from a home-based patient with respect to monitoring by a health care provider treatment facility, comprising:
an activatable “body-condition” test unit configured for personal recognition and interactive telecommunication with the health care provider treatment facility; and
a patient-personalized, screen displayed, patient-respected, computer generated avatar figure, to visually and audibly prompt and guide proper patient responsive self-treatment.

2. The health care monitoring system as recited in claim 1, wherein the “body-condition” test unit comprises a test unit for measuring patient self-administered blood oxygen levels.

3. The health care monitoring system as recited in claim 1, wherein the “body-condition” test unit comprises a test unit for measuring patient self-administered blood pressure test.

4. The health care monitoring system as recited in claim 1, wherein the “body-condition” test unit comprises a test unit for measuring patient self-administered body temperature.

5. The health care monitoring system as recited in claim 1, wherein the “body-condition” test unit comprises a test unit for measuring patient self-administered glucose levels.

6. The health care monitoring system as recited in claim 1, wherein the avatar figure is generated by a computer source within the monitoring system.

7. The health care monitoring system as recited in claim 6, wherein the computer source maintains a variable avatar collection to permit the avatar to show multiple stages of urgency to a patient utilizing the system, as required when patient compliance is not properly completed with certain preselected time intervals.

8. The health care monitoring system as recited in claim 7, wherein the avatar is selected from a group consisting of: one or more of the patient's family members, the patient's medical support team, a religious figure, a historical figure, a political figure, a sport's figure, a friend, and a cartoon character.

9. A health care monitoring and control system for the patient-responsive/non-responsive treatment and reporting from a home-based patient with respect to a health care provider treatment facility, consisting of:
a patient/system activated “body-condition” test unit configured for personal recognition and interactive telecommunication with the health care provider treatment facility; and
a patient-personalized, screen displayed, patient-respected computer generated avatar figure, to prompt and guide proper patient treatment, wherein the avatar is selected from a group consisting of: one or more of the patient's family members, the patient's medical support team, a religious figure, a historic figure, a sport's figure, and a cartoon character.

10. A method of providing health care monitoring and health care support by a home based monitoring and healthcare support system comprising:
arranging a patient-specific, patient-personalized, avatar-pre-programmed healthcare monitoring and control system for in-home use by a specific health impaired patient;
initiating the system monitoring and support of a patient using the system, by introduction of a patient's limb through a patient-receiving annular blood pressure monitoring cuff;
guiding the patient through the monitoring and support procedure by a patient-identifiable avatar pre-selected by the patient/healthcare provider, and represented on a monitor of the system; wherein the patient identifiable avatar is pre-selected by the patient from the group consisting of: one or more of the patient's family members, the patient's medical support team, a religious figure, a historic figure, a sport's figure, and a cartoon character, and wherein the healthcare monitoring system is in electronic communication and monitored by a professional healthcare agency;
testing the patient's blood by a finger clip oximeter for an oximeter reading prior to reading the patient's blood pressure by the blood pressure monitoring cuff;
testing the patient's blood pressure subsequent to the oximeter reading as prompted and instructed by the particular avatar character;
reporting the results of the system's testing to a healthcare provider via an internet communication therewith;
elevating the urgency expressed by the avatar in communication with the patient undergoing the test procedures if the system or the healthcare provider determines an improper or undesired result or condition has occurred;
changing the avatar from one initially utilized to a different more forceful avatar should the system indicate a need to do so by its computer; and
changing the avatar from one initially utilized by the system and shown on a monitor to the patient, to a different more forceful avatar shown subsequently to the patient on a monitor, should the system indicate a need to do so by the healthcare provider.

11. A method of providing health care monitoring and health care support by a home based monitoring and healthcare support system in the house of a patient, comprising:
arranging a patient-specific, patient-personalized, avatar-pre-programmed healthcare monitoring and control system for in-home use by a specific health impaired patient;
initiating the system monitoring and support of a patient using the system, by introduction of a patient's limb through a patient-receiving annular blood pressure monitoring cuff; and
guiding the patient through the monitoring and support procedure by a patient-identifiable, avatar pre-selected by the patient/healthcare provider, and represented on a monitor of the system.

12. The method as recited in claim 11, wherein the patient identifiable avatar is pre-selected by the patient from the group consisting of: one or more of the patient's family members, the patient's medical support team, a religious figure, a historic figure, a sport's figure, and a cartoon character.

13. The method as recited in claim 11, wherein the healthcare monitoring system is in electronic communication and monitored by a professional healthcare agency.

14. The method as recited in claim 11, including:
testing the patient's blood by a finger clip oximeter for an oximeter reading prior to reading the patient's blood pressure by the blood pressure monitoring cuff.

15. The method as recited in claim 14, including:
testing the patient's blood pressure subsequent to the oximeter reading as prompted and instructed by the particular avatar character.

16. The method as recited in claim 15, including:
reporting the results of the system's testing to a healthcare provider via an internet communication therewith.

17. The method as recited in claim 15, including:
elevating the urgency expressed by the avatar in communication with the patient undergoing the test procedures if the system or the healthcare provider determines an improper or undesired result or condition has occurred.

18. The method as recited in claim 17, including:
changing the avatar from one initially utilized, to a different more forceful avatar should the system indicate a need to do so by the patient's response sensed by the system computer.

19. The method as recited in claim 18, including:
changing the avatar from one initially utilized by the system and shown on a monitor to the patient, to a different more forceful avatar shown subsequently to the patient on a monitor, should the system indicate a need to do so by the healthcare provider.

20. The method as recited in claim 11, wherein the system includes satellite monitors selected from the group consisting of: wireless monitors in further rooms within the house of the patient, wireless monitors worn by the patient for receipt of avatar instruction.
